(19) United States
(12) Patent Application Publication
(10) Pub. No.: US 2026/0325848 A1
(43) Pub. Date: Oct. 22, 2026

(54) MEASUREMENT OF EVOKED RESPONSES TO NEURAL STIMULATION

(71) Applicant: Saluda Medical Pty Limited, Macquarie Park (AU)

(72) Inventors: Zubin Zarir NANAVATI, Macquarie Park (AU); Peter Scott Vallack SINGLE, Macquarie Park (AU)

(21) Appl. No.: 19/654,844

(22) Filed: Apr. 22, 2026

(30) Foreign Application Priority Data
Apr. 22, 2025 (AU) 2025901407

(51) Int. Cl. A61N 1/36; A61N 1/05

(52) U.S. Cl. CPC A61N 1/36139; A61N 1/0551; A61N 1/36071

(57) ABSTRACT

Disclosed is an implantable device for controllably delivering neural stimuli to a neural pathway. The device comprises a pulse generator to deliver neural stimuli according to a stimulation set. This stimulation set includes one or more implanted stimulation electrodes and one or more stimulus parameters. The device comprises measurement circuitry to capture a signal, sensed on the neural pathway by one or more implanted measurement electrodes. A controller is configured to electrically connect the stimulation electrodes defined by a first stimulation set with those defined by a second stimulation set, control the stimulus source to deliver a first neural stimulus according to the first stimulation set, and control the source to deliver a second neural stimulus according to the second stimulation set. The controller captures a signal sensed on the neural pathway and measures, from the signal, a characteristic of the neural response evoked by the second neural stimulus.

The present application claims priority from Australian Provisional Patent Application No. 2025901407 filed on 22 Apr. 2025, the contents of which are incorporated herein by reference in their entirety.

### TECHNICAL FIELD

The present invention relates to neural stimulation and in particular to methods and devices for measurement of evoked responses to neural stimulation in the presence of artefact.

### BACKGROUND OF THE INVENTION

There are a range of situations in which it is desirable to apply neural stimuli in order to alter neural function, a process known as neuromodulation. For example, neuromodulation is used to treat a variety of disorders including chronic pain, movement disorders, and voiding disorders. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either inhibitory or excitatory effects on neural networks. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal). Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. The spinal cord itself moves within the cerebrospinal fluid (CSF) with respect to the dura and the electrode array. During postural changes, the distance between the spinal cord and the electrode array can change significantly. This effect is so large that postural changes alone can cause a previously comfortable and effective stimulus regime to become either ineffectual or painful.

Another control problem facing neuromodulation devices of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable devices, increased power consumption results in more frequent charging and, given that batteries only permit a limited number of charging cycles, this ultimately reduces the implanted lifetime of the device.

Attempts have been made to address such problems by way of feedback or closed-loop control, such as using the methods set forth in International Patent Publication No. WO2012/155188 by the present applicant, the content of which is incorporated herein by reference. Feedback control seeks to compensate for relative nerve/electrode movement by controlling the intensity of the delivered stimuli to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in stimulus artefact, which may manifest in the sensed signal as a decaying output of the order of several millivolts after the end of the stimulus. As the neural response can be contemporaneous with the stimulus crosstalk or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result in a time-decaying artefact waveform of positive or negative polarity, their identification and elimination can be laborious.

Evoked neural responses are less difficult to measure when they appear later in time than the artefact, or when the signal-to-artefact ratio is sufficiently high. The artefact decays over a time of 1 to 2 ms after the stimulus and so, provided the neural response is measured after this time window, a neural response measurement can be more easily obtained. This is the case in surgical monitoring where there are large distances (e.g. more than 12 cm for nerves conducting at 60 ms⁻¹) between the stimulus and measurement electrodes so that the propagation time from the stimulus site to the measurement electrodes exceeds 2 ms, which is longer than the typical duration of stimulus artefact.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity (e.g. less than 5 cm). In such situations the measurement process must overcome artefact directly.

In a feedback-controlled implantable neuromodulation device, it is beneficial to have a wide choice of measurement electrodes among the electrodes in the implanted array. Ideally any electrode should be available to be selected as a measurement electrode, so that the best quality signal may be sensed regardless of which electrodes are used to stimulate the neural tissue. For example, a measurement electrode at the opposite end of the array to the stimulation electrode may yield the highest quality sensed signals on account of the delay between the stimulus and the time of arrival of the evoked response. Such a delay increases the separation in time between artefact and evoked response. In a feedback-controlled implantable neuromodulation device programmed to synchronously deliver multiple interleaved pulse trains at different stimulation electrodes, such a choice of measurement electrode may mean that the optimally-located measurement electrode for a given pulse train has recently been used as a stimulation electrode for a previous pulse train (a situation referred to as role overlap). Artefact may therefore occur at the measurement electrode as a result of the stimuli delivered according to the previous pulse train. Such artefact is referred to as overlap artefact. It is therefore important that overlap artefact is mitigated to enable electrodes subject role overlap to be used successfully as measurement electrodes.

Any discussion of documents, acts, materials, devices, articles or the like which has been included in the present Background is solely for the purpose of providing a context for the present technology. It is not to be taken as an admission that any or all of these matters form part of the prior art base or were common general knowledge in the field relevant to the present technology as it existed before the priority date of each claim of the present disclosure.

### SUMMARY OF THE INVENTION

The present invention seeks to provide a neural stimulation device, which will overcome or substantially ameliorate at least some of the deficiencies of the prior art, or at least provide an alternative.

According to a first aspect of the present technology, there is provided an implantable device that may include a pulse generator configured to deliver neural stimuli, according to a stimulation set, to a neural pathway of a patient, the neural stimuli being configured to evoke neural responses from the neural pathway, where the stimulation set may define one or more implanted stimulation electrodes and one or more stimulus parameters. The implantable device may also include measurement circuitry configured to capture a signal sensed on the neural pathway, during a signal window, by one or more implanted measurement electrodes. The implantable device may further include a controller configured to: electrically connect together the stimulation electrodes defined by a first stimulation set and the stimulation electrodes defined by a second stimulation set; control the pulse generator to deliver a first neural stimulus according to the first stimulation set; control the pulse generator to deliver a second neural stimulus according to the second stimulation set; control the measurement circuitry to capture a signal sensed on the neural pathway during a signal window subsequent to delivery of the second neural stimulus; and measure, from the signal, a characteristic of a neural response evoked by the second neural stimulus.

Implementations may include one or more of the following features. The controller may be further configured to: adjust, using a feedback controller, a stimulus parameter of the one or more stimulus parameters defined by the second stimulation set based on the measured characteristic. is the controller may be further configured to repeatedly carry out the connecting, controlling, measuring, and adjusting so as to maintain the measured characteristic at or near a target value. The controller may be further configured to adjust a stimulus parameter of the one or more stimulus parameters defined by the first stimulation set based on the adjustment to the stimulus parameter of the one or more stimulus parameters defined by the second stimulation set. The first neural stimulus may be an asymmetric triphasic stimulus pulse. Electrically connecting together the the stimulation electrodes defined by the first stimulation set and the stimulation electrodes defined by the second stimulation set may comprise electrically connecting together all the implanted stimulation electrodes

Implementations of the described techniques may include a method or process. Aspects of the present disclosure provide a method of controllably delivering neural stimuli to a neural pathway of a patient via a plurality of implanted stimulation electrodes. In one general aspect, the method may comprise electrically connecting together the stimulation electrodes defined by a first stimulation set and the stimulation electrodes defined by a second stimulation set, wherein each stimulation set may define one or more of the implanted stimulation electrodes and one or more stimulus parameters. The method may further comprise delivering a first neural stimulus to the neural pathway of the patient, the neural stimulus being delivered according to the first stimulation set. The method may further comprise delivering a second neural stimulus to the neural pathway of the patient, the neural stimulus being delivered according to the second stimulation set. The method may further comprise capturing a signal sensed on the neural pathway, during a signal window subsequent to delivering the second neural stimulus. The method may comprise measuring, from the signal, a characteristic of a neural response evoked by the second neural stimulus.

The method may further comprise adjusting a stimulus parameter of the one or more stimulus parameters defined by the second stimulation set based on the measured characteristic. The method may further comprise repeatedly carrying out the connecting, delivering, capturing, measuring, and adjusting so as to maintain the measured characteristic at or near a target value. The method may further comprise adjusting a stimulus parameter of the one or more stimulus parameters defined by the first stimulation set based on the adjustment to the stimulus parameter of the one or more stimulus parameters defined by the second stimulation set.

Other implementations of this aspect include corresponding computer systems, apparatus, and computer programs recorded on one or more computer storage devices, each configured to perform the actions of the methods.

The present technology has been developed primarily for use in or with neuromodulation of the spinal cord and will be described hereinafter mostly with reference to this application. However, it will be appreciated that the present technology is not limited to this particular field of use, and may be applied in other neuromodulation contexts, including but not limited to sacral nerve stimulation, pudendal nerve stimulation, deep brain stimulation, stimulation of other parts of the peripheral and central nervous system. It will further be appreciated that the present technology may be applied for treatment of conditions other than chronic pain, including but not limited to movement disorders, Crohn's disease, rheumatoid arthritis, diabetes, Reynaud's phenomenon, pelvic floor disorders, chronic inflammatory conditions, migraine, stroke, or depression.

### BRIEF DESCRIPTION OF THE DRAWINGS

Notwithstanding any other implementations which may fall within the scope of the present invention, one or more implementations of the present invention will now be described, by way of example only, with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a bundle of target nerve fibres;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system implementable by the electronics module of FIG. 2, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 illustrates the stimulus pulses delivered by a stimulation program with four interleaved stimulation sets (stimsets);

FIG. 9 is a schematic illustrating elements and inputs of a multi-stimset closed-loop neural stimulation (CLNS) system implementable by the electronics module of FIG. 2, according to one implementation of the present technology;

FIG. 10a illustrates role overlap in the multi-stimset CLNS system of FIG. 9;

FIG. 10b illustrates role overlap in the multi-stimset CLNS system of FIG. 9;

FIG. 11 illustrates a multi-stimset program comprising two stimsets and a shorting pulse for each stimset;

FIG. 12 illustrates a multi-stimset program comprising two stimsets and a single shorting pulse according to one aspect of the present technology;

FIG. 13 illustrates the stimulus pulses delivered by a stimulation program with four interleaved stimsets according to one implementation of a further aspect of the present technology;

FIG. 14 illustrates an asymmetric cathodic-first triphasic stimulus pulse;

FIG. 15 is a graph that plots the amount of overlap artefact against the ISI, utilising the single shorting pulse at the start of each cycle as illustrated in FIG. 12;

FIG. 16 illustrates a multi-stimset program comprising two stimsets and a shorting pulse for each stimset according to a further aspect of the present technology;

FIG. 17a illustrates a single-stimset program according to a further aspect of the present technology; and

FIG. 17b illustrates a single-stimset program according to a further aspect of the present technology.

### DETAILED DESCRIPTION OF THE PRESENT TECHNOLOGY

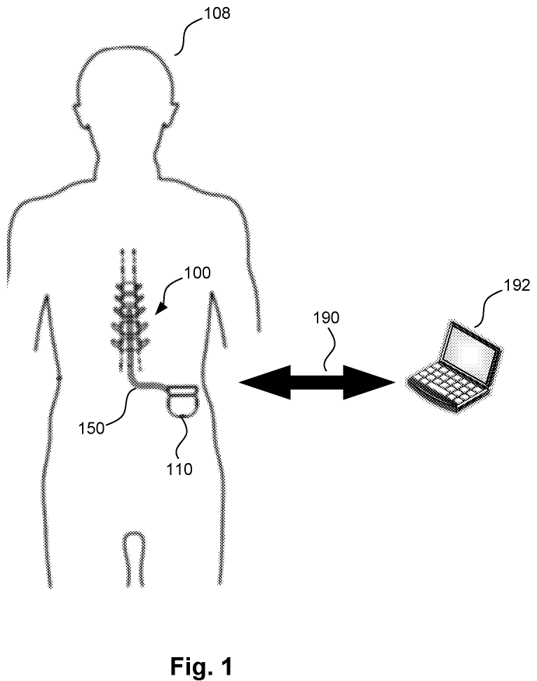

FIG. 1 schematically illustrates an implanted spinal cord stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly, typically comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

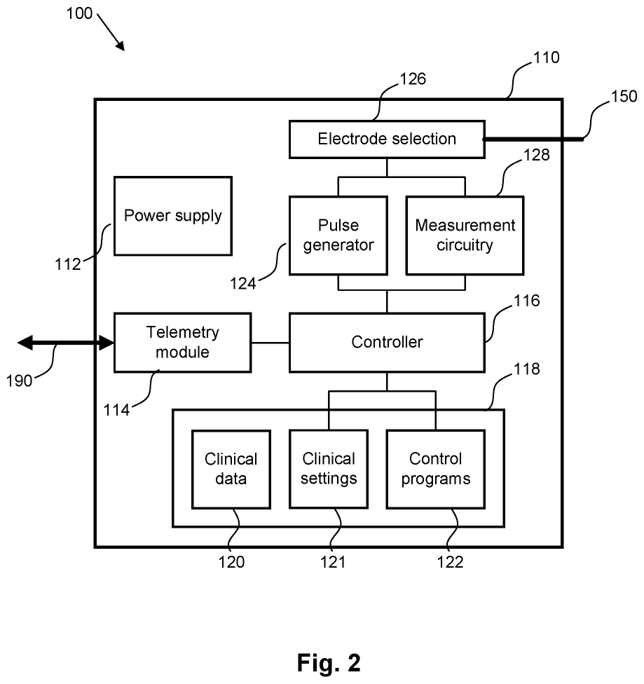

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier or an analog-to-digital converter (ADC), is configured to process signals comprising neural responses sensed by measurement electrode(s) of the electrode array 150 as selected by electrode selection module 126.

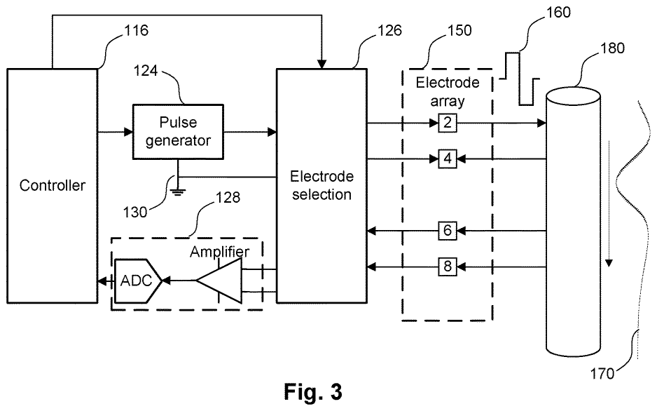

FIG. 3 is a schematic illustrating interaction of the implanted stimulator 100 with a bundle of target nerve fibres 180 in the patient 108. In the implementation illustrated in FIG. 3 the target fibres 180 may be located in the spinal cord, however in alternative implementations the stimulator 100 may be positioned adjacent any target neural tissue including a peripheral nerve, visceral nerve, sacral nerve, parasympathetic nerve, or a brain structure. Electrode selection module 126 selects a stimulus electrode 2 of electrode array 150 through which to deliver a pulse from the pulse generator 124 to surrounding neural tissue including target fibres 180. A pulse may comprise one or more phases, e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode array 150.

Stimulation electrodes refer to the set of one or more stimulus electrodes and the one or more return electrodes. Stimulation electrodes may comprise a single electrode. The set of stimulation electrodes may be referred to as a stimulus electrode configuration (SEC). Electrode selection module 126 is illustrated as connecting to a ground 130 of the pulse generator 124 to enable stimulus current return via the return electrode 4. However, other connections for current return may be used in other implementations.

Delivery of an appropriate stimulus via stimulation electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of a spinal cord stimulator for chronic pain may be associated with paresthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that may be experienced by the patient as paresthesia. When a stimulus electrode configuration is found that evokes paresthesia in a location and of a size which is congruent with the area of the patient's body affected by pain and of a quality that is comfortable for the patient, the clinician or the patient nominates that configuration for ongoing use. The therapy parameters may be loaded into the memory 118 of the electronics module 110 as the clinical settings 121.

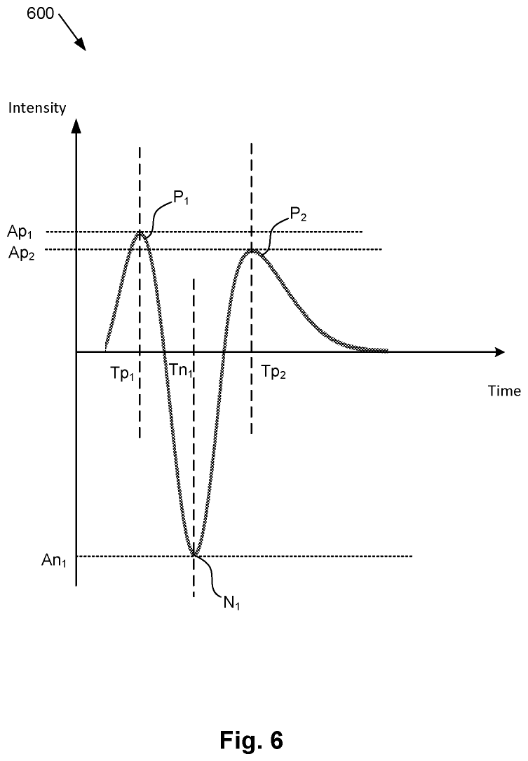

FIG. 6 illustrates the typical form of an ECAP 600 of a healthy subject, as sensed by a single measurement electrode referenced to the system ground 130 or referenced to an indifferent electrode. Such configurations are referred to as single-ended ECAP measurement. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms. Characteristics of an evoked neural response (e.g., characteristics of an ECAP) may comprise one or more of, but not limited to: an amplitude of a first positive peak P1; an amplitude of a second positive peak P2; an amplitude of the negative peak N1; and a peak-to-peak or root-mean-square amplitude of the ECAP.

The stimulator 100 is further configured to measure the intensity of ECAPs 170 propagating along target fibres 180, whether such ECAPs are evoked by the stimulus from electrodes 2 and 4, or otherwise evoked. To this end, any electrodes of the electrode array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration (MEC). The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (uV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, target response intensity, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

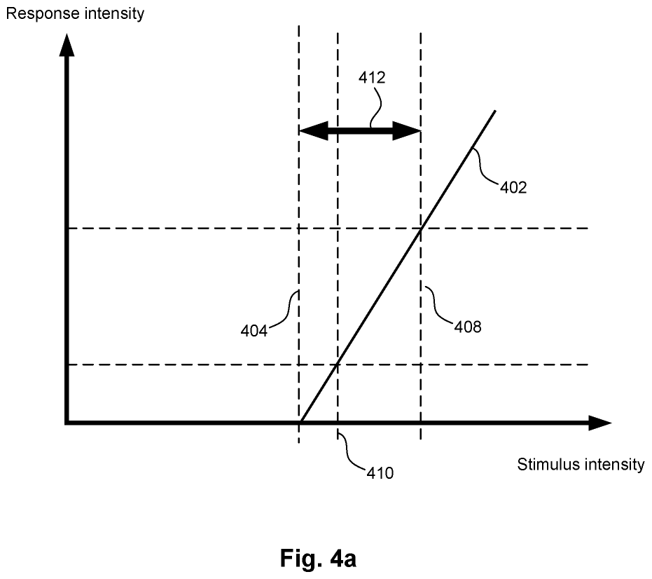

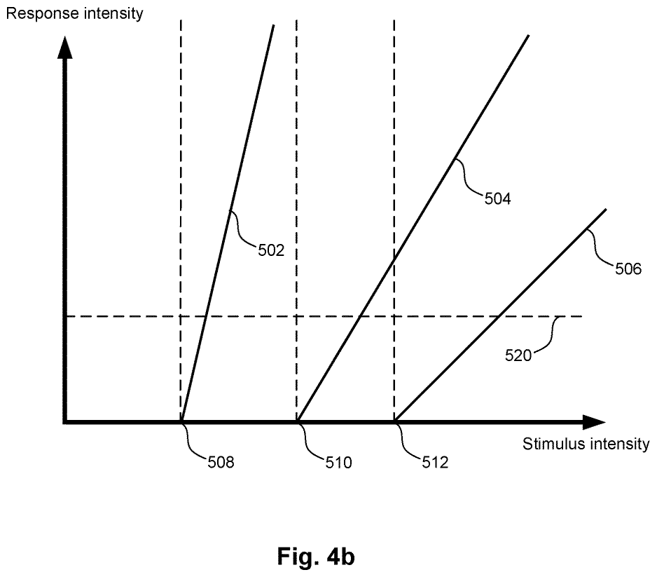

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled in piecewise linear form as:

$$d = \{ \begin{matrix}S ( s - T ) , & s \geq T \\ 0 , & s < T\end{matrix} \qquad ( 1 )$$

where s is the stimulus intensity, dis the ECAP amplitude, Tis the ECAP threshold and Sis the slope of the activation plot (referred to herein as the patient sensitivity) above the ECAP threshold T. The sensitivity S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 is a value of stimulus intensity that corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal-to-noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the spinal cord. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the patient sensitivity also changes, as indicated by the varying slopes of activation plots 502, 504, and 506. In general, as the distance between the stimulus electrodes and the spinal cord increases, the ECAP threshold increases and the sensitivity decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at or near a target response intensity. For example, the device may calculate an error between a target ECAP amplitude and a measured ECAP amplitude, and adjust the applied stimulus intensity to bring the measured ECAP amplitude closer to the target ECAP amplitude, such as by adding the scaled error to the current stimulus intensity. A neuromodulation device that operates by adjusting the applied stimulus intensity to maintain a feedback variable at or near a target value is said to be operating in closed-loop mode and will also be referred to as a closed-loop neural stimulation (CLNS) device. By adjusting the applied stimulus intensity to maintain the measured ECAP amplitude at or near an appropriate target response intensity, such as a target ECAP amplitude 520 illustrated in FIG. 4b, a CLNS device will generally keep the stimulus intensity within the therapeutic range as patient posture varies.

A CLNS device comprises a pulse generator that takes a stimulus intensity value and converts it into neural stimuli comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the pulse generator to implement the received stimulus intensity value. For example, all stimulus parameters may be held constant except stimulus amplitude which is determined in proportion to the received stimulus intensity value. Alternatively, all stimulus parameters may be held constant except pulse width which is determined in proportion to the received stimulus intensity value.

In an example CLNS system, the user sets a target response intensity, and the CLNS device performs proportional-integral-differential (PID) control. In some implementations, the differential and proportional contributions are disregarded and the CLNS device uses a first order integrating feedback loop. The pulse generator generates a stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target response intensity is properly chosen, the patient receives consistently comfortable and therapeutic stimulation through posture changes and other perturbations to the stimulus/response behaviour.

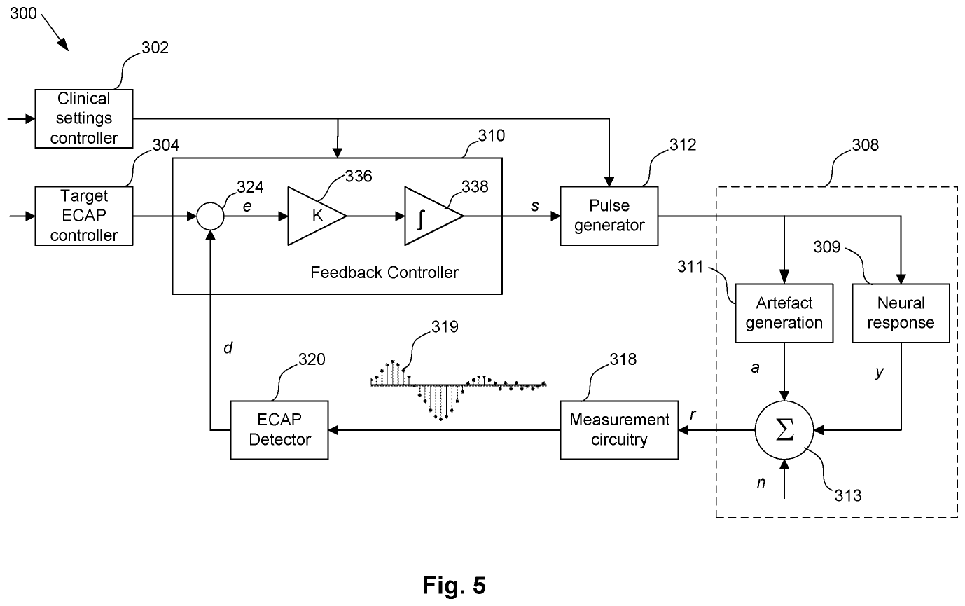

FIG. 5 is a schematic illustrating elements and inputs of a closed-loop neural stimulation (CLNS) system 300, implementable in part by the electronics module 110 of FIG. 2, according to one implementation of the present technology. The system 300 comprises a pulse generator 312 which converts a stimulus intensity parameter s, in concert with a set of predefined stimulus parameters, into neural stimuli comprising a sequence of electrical pulses delivered via the stimulation electrodes (not shown in FIG. 5). According to one implementation, the predefined stimulus parameters comprise the number and order of phases, the number of stimulus electrode poles, the pulse width, and the stimulus rate or frequency, and the pulse generator 312 determines the stimulus amplitude in proportion to the stimulus intensity parameter s.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal to capture a “signal window” 319 comprising a predetermined number of samples of the amplified sensed signal. The ECAP detector 320 processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

s=∫Kedt (2)
where K is the gain of the gain element 336 (the controller gain). This relation may also be represented as

δs=Ke (3)
where δs is an adjustment to the current stimulus intensity parameter s.

A target ECAP amplitude is input to the feedback controller 310 via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the pulse generator 312. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the pulse generator 312 generates a stimulus in accordance with the adjusted stimulus intensity s. Accordingly, there is a delay of one stimulus clock cycle before the stimulus intensity delivered to the patient is updated in light of the error value e.

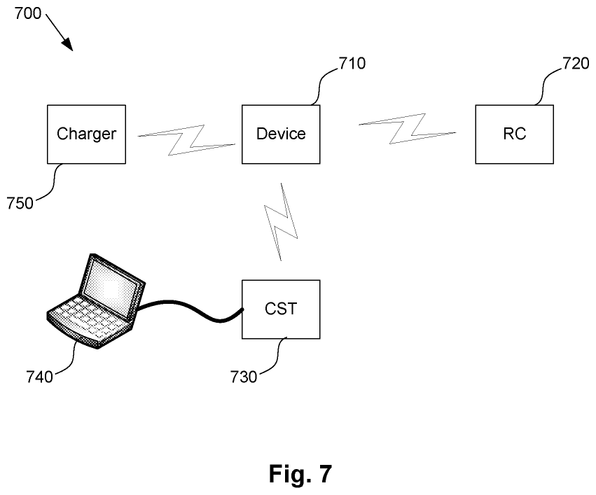

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Multi-Stimset Neural Stimulation

For some patients, it is beneficial for a neural stimulation therapy program to comprise multiple stimulation sets. A stimulation set (“stimset”) defines a stimulus electrode configuration (SEC), along with the stimulus parameters that govern the stimulation pulses delivered via that SEC. In some implementations, a stimset comprises one or more shorting events defined by shorting pulse parameters, as defined herein.

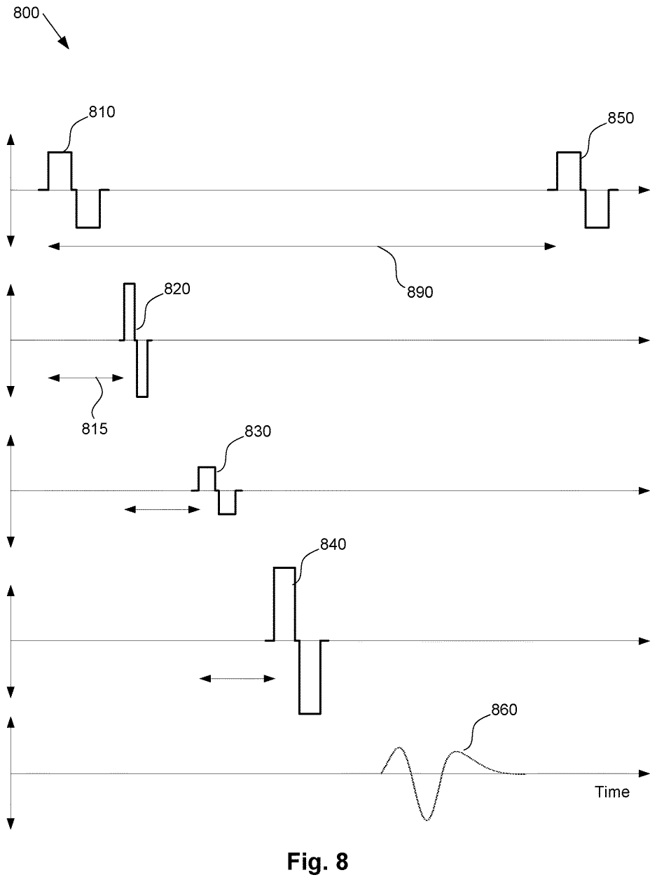

FIG. 8 illustrates stimulus pulses delivered by a stimulation program 800 with four interleaved stimsets. The stimulus pulse train delivered according to each stimset is illustrated on a separate, but vertically aligned, horizontal axis representing time. The vertical axes represent stimulus intensity. All the stimulus pulse trains are delivered at the same stimulus frequency. (It is not a requirement that all the stimulus pulse trains for the respective stimsets are delivered at the same stimulus frequency; however, it is so represented in FIG. 8 for ease of illustration.) The first stimulus pulse 810, delivered according to the first stimset, is illustrated as a biphasic, anodic-first stimulus pulse, though many other stimulus pulse types are contemplated. The second, third, and fourth stimulus pulses 820, 830, and 840, delivered according to the second, third, and fourth stimsets in the program respectively, are also biphasic, anodic-first stimulus pulses with different pulse widths and different amplitudes. Each stimulus pulse is illustrated as delayed in time by a constant amount (the inter-stimulus interval, or ISI, 815) from the stimulus pulse delivered according to the preceding stimset. However, this is not to be interpreted as limiting, since the intervals between the pulses in the various stimsets may be different. Because all the stimulus pulse trains in FIG. 8 are delivered at the same stimulus frequency, the four stimulus pulses 810, 820, 830, 840 form a cycle that repeats indefinitely without any change to the relative timing of the pulses from the different stimsets. The fifth stimulus pulse 850 is a subsequent pulse in the pulse train delivered according to the first stimset and is therefore illustrated on the same time axis as the first stimulus pulse 810, and the cycle repeats thereafter. The stimulus period 890 is the period of repetition of the full cycle and is equal to the reciprocal of the stimulus frequency. In one implementation, the ISI 815 is the stimulus period divided by the number of stimsets, so that the stimuli are evenly spaced throughout the stimulus period 890.

Also illustrated is an evoked neural response in the form of an evoked compound action potential (ECAP) 860 as sensed by a predetermined measurement electrode configuration (MEC) on a common time axis with the stimulus pulses. The illustrated ECAP 860 is evoked by the fourth stimulus pulse 840. A closed-loop neural stimulation (CLNS) system programmed with multiple interleaved stimsets, as illustrated in FIG. 8, may be based on measurements of the ECAP 860. That is to say, closed-loop adjustments to the stimulus parameters of all stimsets may all be based on measurements of the ECAP 860 evoked by a single stimset, referred to as the applied stimset. In FIG. 8, the final stimset corresponding to the fourth stimulus pulse 840 in the cycle is the applied stimset.

If the ISI 815 is short, ECAPs evoked by the first three stimulus pulses 810, 820, and 830 are potentially obscured by stimulus crosstalk or artefact from the stimulus pulses 820, 830, and 840. Therefore, if the ISI 815 is short, only the final stimset in the cycle may evoke a measurable ECAP. If the ISI 815 is greater than the refractory period and sufficiently long that ECAPs evoked by the earlier stimsets are not obscured by stimulus crosstalk and artefact from the other stimulus pulses in the cycle, any of the stimsets in the cycle may evoke a measurable ECAP and may therefore be the applied stimset.

FIG. 9 is a schematic illustrating elements and inputs of a multi-stimset CLNS system 900 implementable by the electronics module 110, according to one implementation of the present technology. The multi-stimset CLNS system 900 is the same as the CLNS system 300 of FIG. 5, with like numbers indicating like elements, with the addition of three further stimsets. The noise addition and artefact generation in FIG. 5 have been omitted from FIG. 9 for clarity. The four stimsets are labelled A, B, C, and D and are delivered by pulse generators 312A, 312B, 312C, and 312D (the latter of which corresponds to the pulse generator 312 in the CLNS system 300) according to respective stimulus intensity parameters s_{A}, s_{B}, s_{C}, and s_{D}, and via respective SECs. The pulses delivered by the pulse generators 312A, 312B, 312C, and 312D correspond to the stimulus pulses 810, 820, 830, and 840 of FIG. 8. The neural response y may be evoked by any of stimsets A, B, C, and D, which is why the neural response box 309 is joined by dashed lines to all four pulse generators 312A, 312B, 312C, and 312D in FIG. 9. The applied stimset is the stimset by which the neural response y is evoked.

In the implementation of FIG. 9, the stimulus intensity parameter s_{D} for stimset D is the largest of the four stimulus intensity parameters s_{A}, s_{B}, s_{C} and s_{D} and is the stimulus intensity parameter that is directly adjusted by the feedback controller 310. Stimset D is therefore referred to as the “master” stimset. The stimulus intensity parameter s_{D} of the master stimset D is scaled by ratios R_{A}, R_{B}, and R_{C} to obtain the stimulus intensity parameters s_{A}, s_{B}, and s_{C} for stimsets A, B, and C respectively at the end of each cycle. The stimset ratios R_{A}, R_{B}, and R_{C}, which are all less than or equal to one, are fixed at the ratios of the respective stimulus intensities s_{A}, s_{B}, and s_{C} at which the respective stimsets were originally programmed, to the originally programmed stimulus intensity s_{D} of the master stimset D and form part of the clinical settings 121 of the multi-stimset program. In such an implementation, the stimulus intensity parameters s_{A}, s_{B}, and s_{C} always remain in fixed ratio with the master stimulus intensity parameter s_{D} and with each other. This is referred to as ratiometric adjustment. So, for example, if the originally programmed stimulus intensities were 1 mA, 2 mA, 4 mA, and 6 mA for the four stimsets A, B, C, and D respectively, the stimset ratios R_{A}, R_{B}, and R_{C} are fixed at programming time at ⅙, ⅓, and ⅔ respectively. If, during therapy, the feedback controller 310 adjusts the master stimulus intensity parameter s_{D} to 6.6 mA, the stimulus intensity parameters s_{A}, s_{B}, and s_{C} are automatically adjusted to 1.1 mA, 2.2 mA, and 4.4 mA respectively. The clinical settings controller 302 provides the respective stimulus parameters to the pulse generators 312A, 312B, 312C, and 312D.

It may be seen from FIG. 9 that the adjustments to the stimulus intensity parameters after each stimulus cycle are all in fixed proportion. A ratiometric multi-stimset CLNS system therefore emulates a CLNS system with four separate feedback loops driven by the four stimsets, wherein each loop has the same controller gain. A ratiometric multi-stimset CLNS system is effective to maintain the responses evoked by each stimset at a constant neural response intensity on the condition that when the patient moves to a new posture, the threshold and slope of all activation plots, both for applied and non-applied stimsets, move in a proportional manner. (See FIG. 4b for examples of activation plots for a given stimset in different postures.)

### Inter-Stimset Overlap

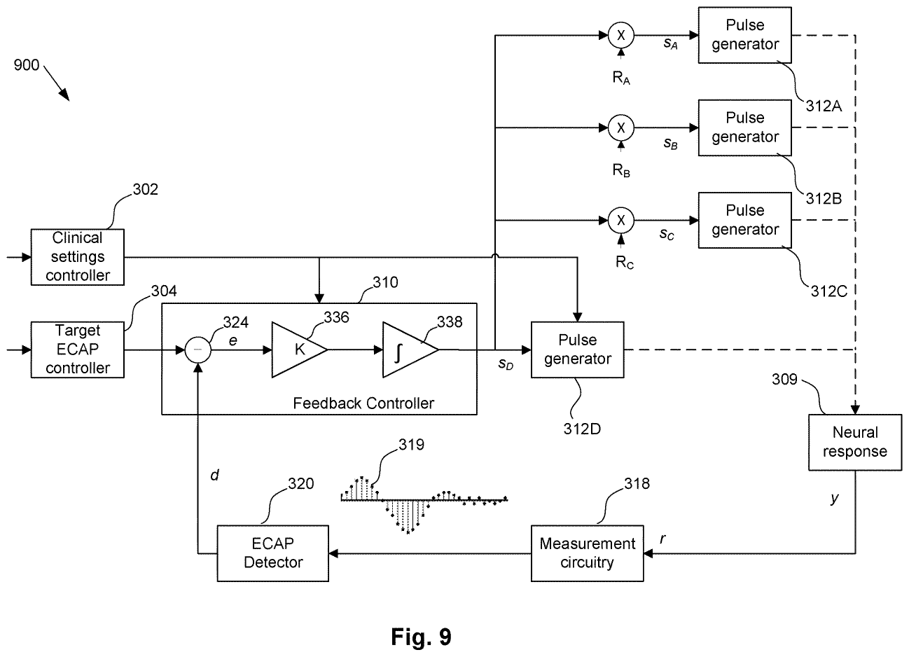

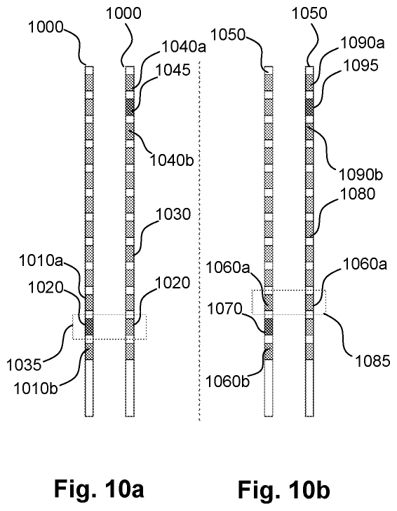

FIGS. 10a and 10b illustrate two situations of role overlap in a multi-stimset CLNS system. Both situations are examples of a species of role overlap referred to herein as inter-stimset overlap. In FIG. 10a, the electrode array 1000, comprising a single percutaneous lead, is depicted in two instances. In the left depiction, a first stimulus is being delivered in tripolar fashion in accordance with a first stimset, comprising stimulus electrode 1020 and return electrodes 1010a and 1010b. In the right depiction of electrode array 1000, a second stimulus is being delivered in tripolar fashion in accordance with a second stimset, comprising stimulus electrode 1045 and return electrodes 1040a and 1040b. Since the second stimset is the applied stimset, a signal is to be sensed subsequent to the stimulus via measurement electrodes 1020 and 1030. Box 1035 indicates that electrode 1020 is subject to inter-stimset overlap, since electrode 1020 acts as a stimulus electrode for the first stimset and a measurement electrode for the second stimset.

In FIG. 10b, the electrode array 1050, comprising a single percutaneous lead, is depicted in two instances. In the left depiction, a first stimulus is being delivered in tripolar fashion in accordance with a first stimset, comprising stimulus electrode 1070 and return electrodes 1060a and 1060b. In the right depiction, a second stimulus is being delivered in tripolar fashion in accordance with a second stimset, comprising stimulus electrode 1095 and return electrodes 1090a and 1090b. Since the second stimset is the applied stimset, a signal is to be sensed, subsequent to the delivery of the stimulus, via measurement electrodes 1080 and 1060a. Box 1085 indicates that electrode 1060a is subject to inter-stimset overlap, since electrode 1060a acts as a return electrode for the first stimset and a measurement electrode for the second stimset.

The interface between a metallic electrode and human tissue may be modelled as a constant-phase element (CPE), as distinct from a capacitor (which is a special case of CPE in which the phase shift is 90 degrees). The bidirectional flow of current during the stimulus pulse through the CPE of an electrode subject to inter-stimset overlap causes subsequent artefact at the input to the measurement circuitry 128 connected to such an electrode. In general (as in FIGS. 10a and 10b), the other measurement electrode is not so overlapped, so different charge dynamics apply at the other input to the measurement circuitry 128, meaning that the artefact is not “common mode” and will be amplified by the differential amplifier in the measurement circuitry 128. This is referred to as inter-stimset overlap artefact.

### Passive Charge Recovery (Electrode Shorting)

A biphasic stimulus pulse, such as the pulse 810 illustrated in FIG. 8, comprises a first phase in which a volume of charge is sourced from a first electrode, passes through the adjacent neural tissue, and is sunk into a second electrode. Then in a second phase, a volume of charge is sourced from the second electrode, passes through the same neural tissue, and is sunk into the first electrode. Because the electrode-tissue interface may be modelled as a CPE, which is quasi-capacitive in character, charge flowing through it tends to build up charge across the interface. In addition, in some stimulators, there is a capacitor referred to as the electrode capacitor between the electrode selection module 126 (FIG. 3) and each electrode. The flow of charge through electrodes during a stimulus phase also builds up a charge across the corresponding electrode capacitors, if present. In principle, as long as the charge delivered with one polarity precisely cancels the charge delivered with the other polarity, no net charge will be added to the CPE or the electrode capacitor (if present) as a result of a stimulus pulse. If, however, there is a mismatch, however slight, between the charges delivered with each polarity, for example due to tolerances of the current sources in the pulse generator 124, a net charge will accumulate across both elements. The resulting polarisation will eventually render the pulse generator 124 unable to deliver the stimulus pulses instructed by the controller 116. Additionally, such accumulated charge may result in an unintended sensation of stimulus if discharged into the neural tissue. To prevent this occurring, the controller 116 may be configured to perform a shorting event. A shorting event comprises the controller 116 electrically connecting (shorting) the stimulation electrodes of an SEC together for a duration referred to as the shorting interval (of time). In some embodiments, the shorting interval may comprise 100 microseconds. Optionally, while shorted together, the stimulation electrodes may also be connected to one of the voltage supply rails (VDDHV or ground) of the pulse generator 124.

During the shorting event, residual charge on the electrode-tissue interface (and the electrode capacitors, if present) resulting from charge mismatch between the phases may be passively recovered by charge balancing. The charge balancing produces a shorting pulse on at least one of the stimulation electrodes.

A shorting event may cause significant artefact at an overlapped electrode, since a shorting event in general, like a stimulus pulse, involves transient current flow through the CPEs of the stimulation electrodes. This current flow may result in artefact if any of the stimulation electrodes is subsequently used as a measurement electrode, as occurs in inter-stimset overlap. This artefact is referred to herein as shorting artefact and can worsen inter-stimset overlap artefact.

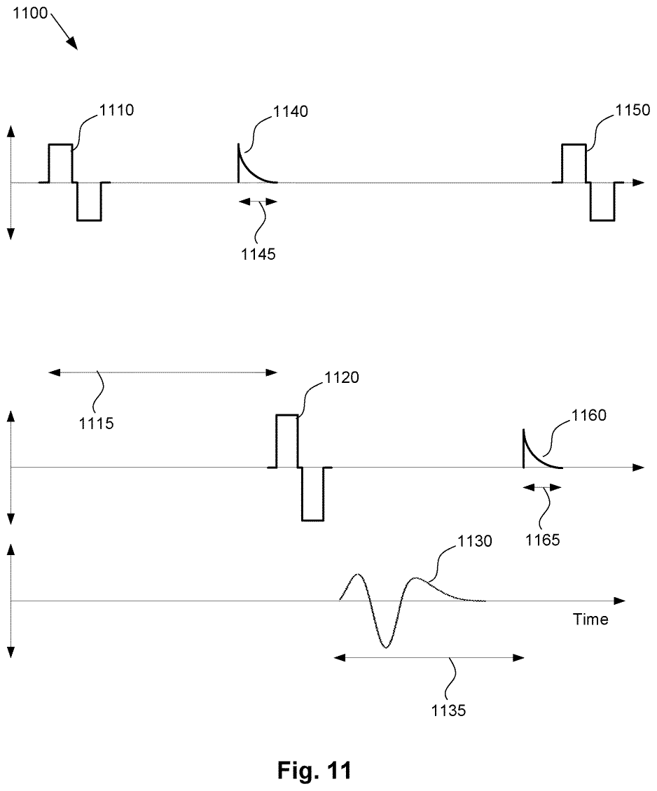

FIG. 11 illustrates a multi-stimset program 1100 comprising two stimsets and a shorting pulse for each stimset. The stimulus pulses 1110 and 1150 are delivered according to the first stimset. The stimulus pulse 1120 is delivered according to the second stimset, which is the applied stimset. There is an inter-stimulus interval 1115 between the pulses, e.g. the pulse 1110, of the first stimset and the subsequent pulses, e.g. the pulse 1120, of the second stimset. The stimulus pulse 1120 evokes the neural response 1130 which may be used to adjust the parameters of both stimsets, as in a multi-stimset CLNS system as described above.

The controller 116 is configured to short together the stimulation electrodes of the first stimset for the duration of a shorting interval 1145. Shorting pulse 1140 occurs in response to the controller 116 shorting together the stimulation electrodes of the first stimset. During the shorting pulse 1140, the magnitude of the current through the shorted stimulation electrodes decays roughly exponentially, as illustrated.

The controller 116 is further configured to short together the stimulation electrodes of the second stimset for the duration of a shorting interval 1165. Shorting pulse 1160 occurs in response to the controller 116 shorting together the stimulation electrodes of the second stimset. The controller 116 is configured to perform the shorting event, such that the shorting interval 1145 ends before the pulse generator delivers stimulus pulse 1120 of the second stimset. In some implementations, the controller 116 is configured to perform the shorting event, such that a resulting shorting artefact commences prior to the signal window 1135, which occurs subsequent to the stimulus pulse 1120 and contains the evoked neural response 1130.

### Optimal Stimulation Sequencing and Charge Balancing for Overlap Artefact Mitigation

One approach to mitigating inter-stimset overlap artefact is to use shorting artefact to counter it. This is possible because the shorting artefact may be of opposite sign to the inter-stimset overlap artefact. Therefore, if any or all of the shorting pulse parameters such as the delay (in relation to the start of the preceding stimulus pulse) and the duration (e.g., shorting interval) of the shorting event are properly configured, the resulting shorting artefact may substantially cancel the inter-stimset overlap artefact at the measurement electrode, subject to inter-stimset overlap.

In methods and devices according to one aspect of the present technology, an alternative approach is taken. Instead of shorting together the stimulation electrodes of each stimset after the delivery of that stimset, there is a single shorting event per complete cycle involving all the stimulation electrodes across all stimsets, with a substantial time interval between the single shorting event and the start of the subsequent signal window. The longer that interval, the less shorting artefact there will be during the subsequent signal window.

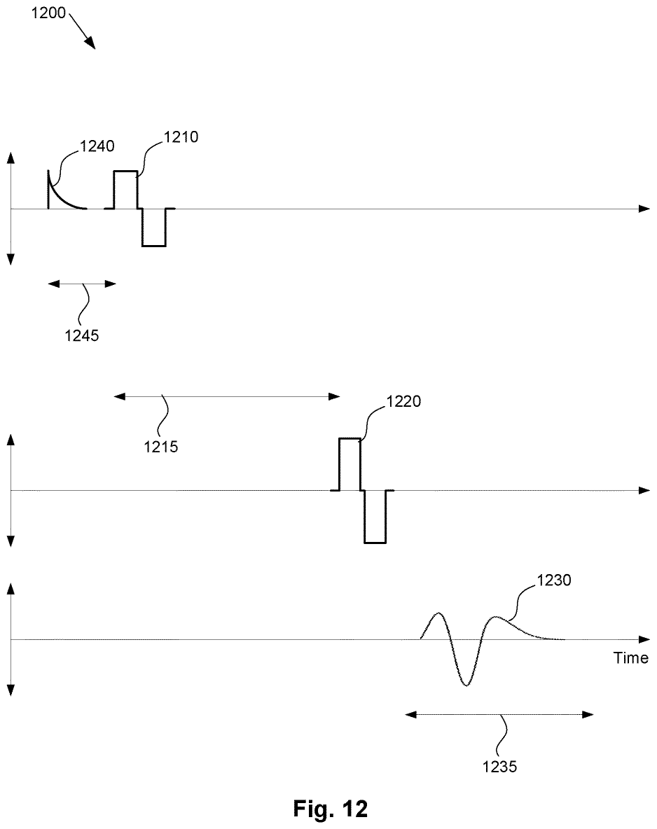

FIG. 12 illustrates a multi-stimset program 1200 comprising two stimsets and a single shorting pulse 1240 according to one aspect of the present technology. The stimulus pulse 1210 is delivered according to the first stimset. The stimulus pulse 1220 is delivered according to the second stimset, which is the applied stimset. There is an inter-stimulus interval 1215 between the pulses, e.g., the pulse 1210, of the first stimset and the subsequent pulses, e.g. the pulse 1220, of the second stimset. The stimulus pulse 1220 evokes the neural response 1230 which may be used to adjust the parameters of both stimsets, as in a multi-stimset CLNS system as described above. In this aspect of the present technology, the stimulation electrodes of all stimsets are shorted together during the single shorting pulse 1240. The controller 116 is configured to commence the shorting pulse 1240 at a predetermined interval 1245 before the stimulus pulse 1210 of the stimset following the applied stimset in each cycle. The predetermined interval 1245, which is one of the shorting pulse parameters for the shorting pulse 1240, is referred to as the post-stimulus shorting delay. In the example of FIG. 12, the post-stimulus shorting delay 1245 is negative, indicating the shorting pulse 1240 takes place before the stimulus pulse 1210. If the applied stimset is the last stimset in the cycle, as in FIG. 12, the stimset following the applied stimset is the first stimset of the next cycle. The controller 116 is therefore configured to perform a shorting event, resulting in the shorting pulse 1240, at the start of each cycle.

In an alternative implementation according to this aspect, the controller 116 is configured to short together all electrodes in the electrode array 150 (not just those electrodes being used as stimulation electrodes for stimsets in the multi-stimset program) during a shorting event, resulting in the shorting pulse 1240. This is beneficial because even passive (unused) electrodes can accumulate charge on their tissue interfaces or electrode capacitors as a result of stimulus pulses due to parasitic effects, and such a shorting event will reduce or eliminate such accumulated charge to reduce the chances of unintended stimulation.

The above-disclosed implementations according to this aspect may be implemented regardless of whether any electrodes are subject to inter-stimset overlap.

In some implementations according to this aspect, the controller 116 may be configured to perform the shorting event at a short predetermined interval after the signal window 1235 containing the evoked neural response 1230, rather than at a predetermined interval before the following stimulus pulse 1210 in the cycle. This timing maximises the interval between the shorting pulse 1240 and the start of the subsequent signal window 1235. In such implementations, the predetermined interval 1245, which is one of the shorting pulse parameters for the shorting pulse 1240, is referred to as the post-window shorting delay and has a positive sign indicating the shorting pulse 1240 takes place after the signal window 1235.

Implementations according to this aspect mitigate inter-stimset overlap artefact by removing the shorting pulses in between the stimsets, which may cause shorting artefact on role-overlapped electrodes that worsens overall artefact. The single shorting pulse 1240, which is timed to occur substantially before the start of the subsequent signal window 1235, passively recovers any imbalance of charge on the electrode capacitors or the electrode-tissue interfaces.

In methods and devices according to a further aspect of the present technology, the stimulus pulses delivered according to the non-applied stimsets are triphasic pulses, rather than biphasic as illustrated in FIG. 8. The applied stimset stimulus pulses remain biphasic as the resulting signal-to-noise ratio on ECAP amplitude measurement is superior to that obtainable from triphasic stimuli. The triphasic pulses may be asymmetric, with the degree of asymmetry configured to mitigate the inter-stimset overlap artefact as described below.

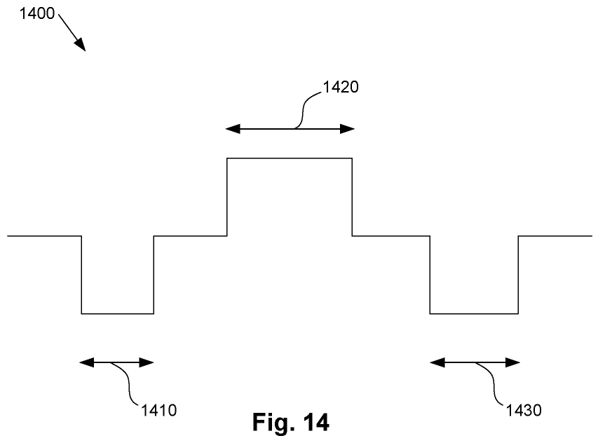

FIG. 14 illustrates an asymmetric cathodic-first triphasic stimulus pulse 1400. The pulse 1400 comprises a first, cathodic phase of duration 1410, a second, anodic phase of duration 1420 with amplitude equal to the amplitude of the first phase, and a third, cathodic phase of duration 1430, also with amplitude equal to the amplitude of the first phase. In order for the pulse 1400 to be charge-balanced, the sum of the durations 1410 and 1430 must equal the duration 1420. However, the durations 1410 and 1430 are in general unequal, which is why the pulse 1400 is termed asymmetric. The ratio of the first duration 1410 to the second duration 1420 (the triphasic ratio) is the degree of asymmetry of the asymmetric triphasic pulse 1400, so that a triphasic ratio of 0.5 indicates symmetric triphasic pulses. An asymmetric anodic-first triphasic stimulus pulse (not illustrated) is the same as the stimulus pulse 1400, except with the three phases reversed in sign from the stimulus pulse 1400.

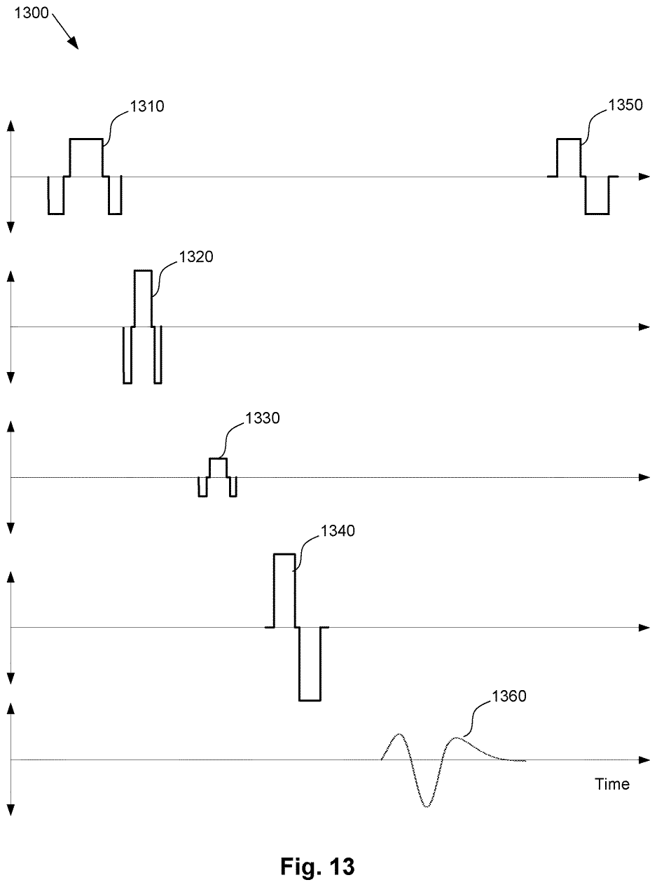

FIG. 13 illustrates stimulus pulses delivered by a stimulation program 1300 with four interleaved stimsets according to one implementation of this aspect of the present technology. The stimulation program 1300 is similar to the stimulation program 800. The first stimulus pulse 1310, delivered according to the first stimset, is illustrated as an asymmetric cathodic-first triphasic pulse, though in some implementations, an asymmetric anodic-first triphasic pulse may be implemented. The second and third stimulus pulses 1320 and 1330, delivered according to the second and third stimsets in the program 1300 respectively, are also asymmetric cathodic-first triphasic pulses. The fourth stimulus pulse 1340, delivered according to the applied stimset, is biphasic like the stimulus pulse 840. The illustrated ECAP 1360 is evoked by the fourth stimulus pulse 1340. The pulse widths and amplitudes of the triphasic stimulus pulses 1310, 1320, and 1330 are all in general different. However, the triphasic ratios of the triphasic stimulus pulses 1310, 1320, and 1330 are equal. In methods and devices according to implementations of this aspect, the common triphasic ratio of the non-applied triphasic stimulus pulses may be configured to mitigate the inter-stimset overlap artefact. In some circumstances the common triphasic ratio may be configured to be 0.5 for optimal mitigation, in which case the triphasic stimulus pulses are symmetric, but in general they will be asymmetric.

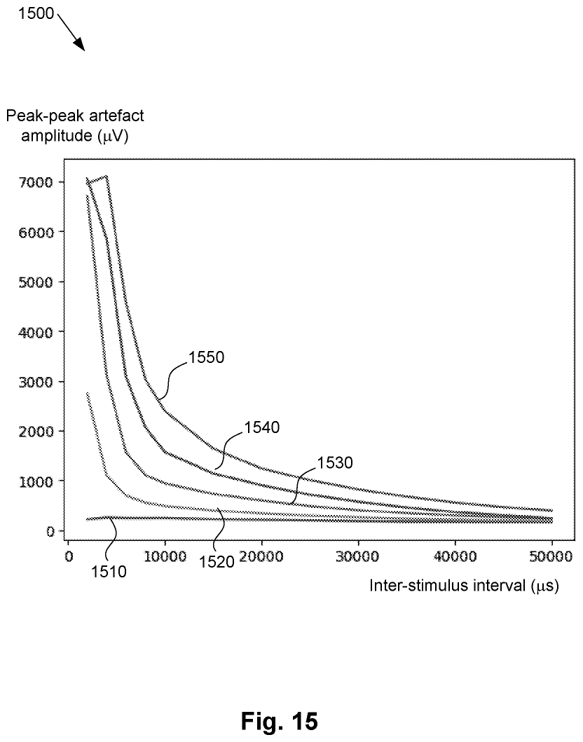

FIG. 15 is a graph 1500 that plots the amount of overlap artefact against the inter-stimulus interval (ISI) of a multi-stimset program which comprises a shorting event, namely the single shorting pulse 1240, at the start of each cycle. The trace 1510, the trace 1520, the trace 1530, the trace 1540, and the trace 1550 plot the peak-to-peak artefact amplitude against ISI at triphasic ratios of 0.45, 0.50, 0.60, 0.75, and 0.90 respectively. It may be seen firstly that the amplitude of artefact decreases as the ISI increases for all values of triphasic ratio, which confirms the artefact is indeed due to inter-stimset overlap, since a greater ISI equates to greater delay between the stimulus pulse and the signal window on the role-overlapped electrode and therefore less artefact. It may also be seen that the least artefact occurs for a triphasic ratio of 0.45, which defines a near-symmetric triphasic pulse. In methods according to this aspect, the magnitude of the artefact may be measured for different triphasic ratios at the selected ISI value (which is governed by the stimulus period), and the triphasic ratio that generates the least artefact may be chosen as the triphasic ratio of each of the non-applied stimsets.

In methods and systems according to a further aspect of the present technology, the order of the stimsets in the stimulation cycle may be altered to mitigate inter-stimset overlap artefact. The aim of the re-ordering is to maximise the interval between the stimulation pulse delivered according to the stimset whose SEC is subject to inter-stimset overlap, and the subsequent signal window. In one implementation according to this aspect, a non-applied stimset subject to inter-stimset overlap is swapped in the order with the non-applied stimset that has the longest interval between the stimulation pulse delivered according to that stimset, and the subsequent signal window. In FIG. 8, that stimset is the first stimset corresponding to the pulse 810, since the applied stimset is the last stimset in the cycle.

In methods and systems according to other aspects of the present technology, other mitigations of inter-stimset overlap artefact include:
Moving the SEC of the non-applied stimset subject to inter-stimset overlap a contact or two along the electrode array, to ensure that no electrodes are subject to inter-stimset overlap. However, care should be taken that this action does not compromise the effectiveness of the therapy.
Widening the tripole of the non-applied stimset subject to inter-stimset overlap. This will, assuming the electrode subject to inter-stimset overlap is a return electrode, also ensure no electrodes are subject to inter-stimset overlap. (This is the case in FIG. 10b, where the electrode 1060a subject to inter-stimset overlap is a return electrode; however, it is not the case in FIG. 10a, where the electrode 1020 subject to inter-stimset overlap is a stimulus electrode.) However, care should be taken that this action does not compromise the effectiveness of the therapy.

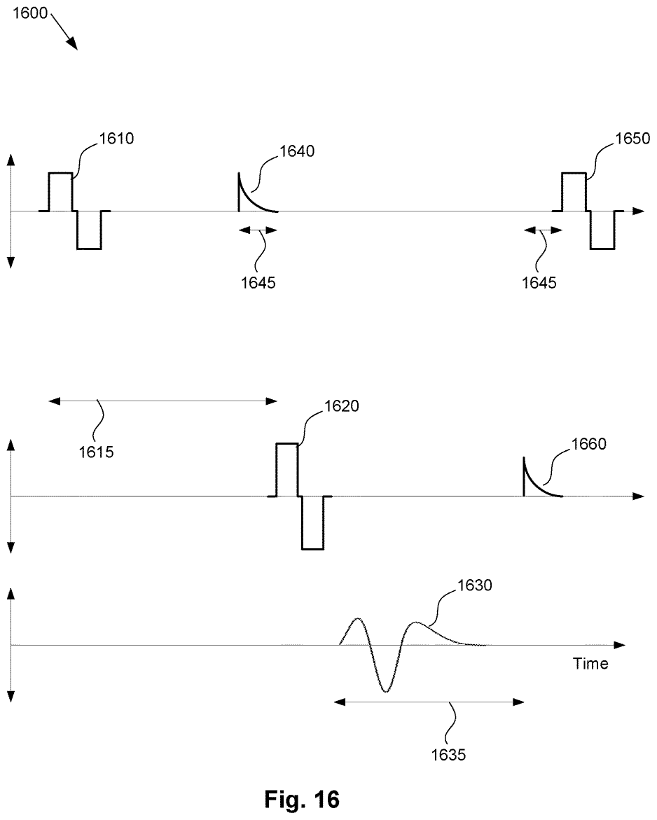

FIG. 16 illustrates a multi-stimset program 1600 comprising two stimsets and a shorting pulse for each stimset, according to a further aspect of the present technology. The stimulus pulses 1610 and 1650 are delivered according to the first stimset. The stimulus pulse 1620 is delivered according to the second stimset, which is the applied stimset. There is an inter-stimulus interval 1615 between the pulses (e.g., the pulse 1610, of the first stimset and the subsequent pulse, e.g. the pulse 1620, of the second stimset). The stimulus pulse 1620 evokes the neural response 1630 during the signal window 1635, which may be used to adjust the parameters of both stimsets, as in a multi-stimset CLNS system as described above. All electrodes in the electrode array 150 are shorted together during the shorting pulse 1640 and during the shorting pulse 1660. The shorting pulse 1640 commences a fixed interval 1645 before the stimulus pulse 1620 of the second stimset. Likewise, the shorting pulse 1640 commences the same fixed interval 1645 before the subsequent stimulus pulse 1650 of the first stimset.

The multi-stimset program 1600 is configured to recover charge imbalance on all electrodes rather than only those used for stimulation, as in FIG. 11. This configuration may be beneficial because, as mentioned above, there may be accumulated charge on the electrode-tissue interfaces or the electrode capacitors of passive (unused) electrodes due to parasitic effects.

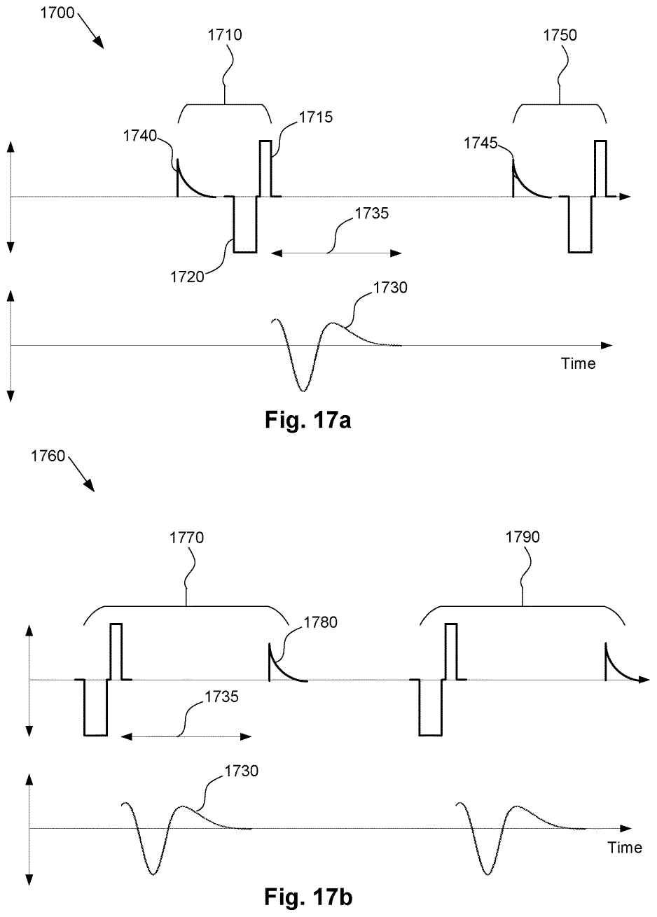

FIG. 17a illustrates a single-stimset program 1700, according to a further aspect of the present technology. The stimulus pulses 1710 and 1750 are delivered according to the first stimset, which is the applied stimset. The stimulus pulse 1710 evokes the neural response 1730 during the signal window 1735. The neural response 1730 may be used to adjust the parameters of the applied stimset, as in a CLNS system as described above. The stimulus pulse 1710 comprises an unbalanced biphasic pulse preceded by a shorting pulse 1740 during which the stimulation electrodes of the first stimset are shorted together. The unbalanced biphasic pulse comprises a cathodic stimulus phase 1720 followed by an anodic charge balance phase 1715 that delivers lesser charge than the cathodic stimulus phase 1720. The unbalanced biphasic pulse, therefore, results in a charge imbalance which persists until recovered by the shorting pulse 1745 of the subsequent stimulus pulse 1750. However, an unbalanced biphasic pulse consumes less power than a fully balanced biphasic pulse, which improves battery life.

Because the program 1700 has only one stimset, there is no role overlap and therefore no inter-stimset overlap artefact or shorting artefact in the signal window 1735. However, there may still be stimulus artefact. The degree of imbalance of the unbalanced biphasic pulse (the ratio of the delivered charge of the anodic charge balance phase 1715 to that of the cathodic stimulus phase 1720) is configured to minimise stimulus artefact in the signal window 1735.

FIG. 17b illustrates a single-stimset program 1760, according to a further aspect of the present technology. The single-stimset program 1760 is similar to the single-stimset program 1700, with like labels indicating like elements. The stimulus pulses 1770 and 1790 are delivered according to the first stimset, which is the applied stimset. The stimulus pulse 1770 evokes the neural response 1730 during the signal window 1735. The neural response 1730 may be used to adjust the parameters of the applied stimset, as in a CLNS system as described above. The stimulus pulse 1770 comprises an unbalanced biphasic pulse followed, after the signal window 1735, by a shorting pulse 1780 during which the stimulation electrodes of the first stimset are shorted together. The unbalanced biphasic pulse of the stimulus pulse 1770 results in a charge imbalance which is recovered by the shorting pulse 1780. The degree of imbalance of the unbalanced biphasic pulse is configured to minimise stimulus artefact in the signal window 1735.

### Interpretation

The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be implemented as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion. The present technology is not limited to any particular programming language or operating system.

### Wireless

In the context of the present disclosure, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of the present disclosure, the term “wired” and its derivatives may be used to describe circuits, devices, systems, methods, techniques, communications channels, etc., that may communicate data through the use of modulated signals propagating through a conductive medium. The term does not imply that the associated devices are coupled by electrically conductive wires.

Wireless communication standards that can be accommodated include IEEE 802.11 wireless LANs and links, Bluetooth, and wireless Ethernet. The technology disclosed herein may be implemented using devices conforming to other network standards and for other applications, including, for example other WLAN standards and other wireless standards such as MICS.

### Processes

Unless specifically stated otherwise, as apparent from the following discussions, it is appreciated that throughout the specification discussions utilizing terms such as “processing”, “computing”, “comparing”, “estimating”, “calculating”, “determining”, “analysing” or the like, refer to the action or processes of a computer or computing system, or similar electronic computing device, that manipulate or transform data represented as physical, such as electronic, quantities into other data similarly represented as physical quantities, or to otherwise execute a predefined procedure suitable to effect the described actions.

### Processor

In a similar manner, the term “processor” may refer to any device or portion of a device that processes electronic data, e.g., from registers or memory, to transform that electronic data into other electronic data that, e.g., may be stored in registers or memory. A “computer” or a “computing device” or a “computing machine” or a “computing platform” may include one or more processors.

The methods described herein are, in one embodiment, performable by one or more processors that accept computer-readable (also called machine-readable) code containing a set of instructions that when executed by one or more of the processors cause the one or more processors to carry out at least one of the methods described herein. Any processor capable of executing a set of instructions (sequential or otherwise) that specify actions to be taken are included within the meaning of the term “processor”. Thus, one example is a typical processing system that includes one or more processors. The processing system further may include a memory subsystem including main RAM or a static RAM, or ROM.

### Networked or Multiple Processors

In alternative embodiments, the one or more processors operate as respective standalone device(s) or may be connected, e.g., networked to other processor(s), in a networked deployment. The one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine may be illustrated, the term “machine” shall also be taken to include any collection of machines that individually or jointly execute a set (or multiple sets) of instructions to perform any one or more of the methods discussed herein.

### Additional Implementations

Thus, one implementation of each of the methods described herein is in the form of a computer-readable medium carrying a set of instructions, e.g., a computer program that are for execution on one or more processors. Thus, as will be appreciated by those skilled in the art, aspects of the present technology may be implemented as a method, an apparatus such as a special purpose apparatus, an apparatus such as a data processing system, or a computer-readable medium. The computer-readable medium carries computer-readable code including a set of instructions that when executed on one or more processors cause the processor or processors to implement a method. Accordingly, aspects of the present technology may take the form of a method, an entirely hardware implementation, an entirely software implementation or an implementation combining software and hardware aspects. Furthermore, the present technology may take the form of a carrier medium (e.g., a computer program product) carrying computer-readable program code embodied in the medium.

### Carrier Medium

The software may further be transmitted or received over a network via a network interface device. While the carrier medium is shown in an example embodiment to be a single medium, the term “carrier medium” should be taken to include a single medium or multiple media (e.g., a centralized or distributed database, or associated caches and servers) that store the one or more sets of instructions. A carrier medium may take many forms, including but not limited to, non-volatile media, volatile media, and transmission media.

### Means for Carrying Out a Method or Function

Furthermore, some of the implementations are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus is an example of a means for carrying out the function performed by the element.

Those of skill would further appreciate that the various illustrative logical blocks, modules, and algorithm steps described in connection with the implementations disclosed herein may be implemented as electronic hardware, computer software running on a special purpose machine that is programmed to carry out the operations described in the present disclosure, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary implementations.

### Implementations

Reference throughout the present disclosure to “one implementation” or “an implementation” means that a particular feature, structure or characteristic described in connection with the implementation is included in at least one implementation of the present technology. Thus, appearances of the phrases “in one implementation” or “in an implementation” in various places throughout the present disclosure are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the present technology, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of the present technology.

Furthermore, while some implementations described herein include some, but not other features included in other implementations, combinations of features of different implementations are meant to be within the scope of the present technology, and form different implementations of the present technology, as would be understood by those in the art. For example, in the following claims, any of the claimed implementations can generally be used in any combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude or position to indicate that the value or position described is within a reasonable expected range of values or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that each value between two particular values is also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicates that different instances of like objects are being referred to, and is not intended to imply that the objects so described must be in a given sequence, either temporally, spatially, in ranking, or in any other manner.

### Specific Details

In the description provided herein, numerous specific details are set forth. However, it is understood that implementations of the present technology may be practiced without these specific details. In other instances, well-known methods, structures and techniques have not been shown in detail in order not to obscure an understanding of the present technology.

### TERMINOLOGY

Throughout the present disclosure, the terms “a” and “an” mean “one or more”, unless expressly specified otherwise.

Throughout the present disclosure, the word “comprise”, or variations such as “comprises” or “comprising”, will be understood to imply the inclusion of a stated element, integer, or step, or group of elements, integers, or steps, but not the exclusion of any other element, integer, or step, or group of elements, integers, or steps.

Throughout the present disclosure, a statement that an element may be “at least one of” or “one or more of” a list of options is to be understood to mean that the element may be any one of the listed options, or may be any combination of two or more of the listed options.

Throughout the present disclosure, the word “or” is to be read inclusively rather than exclusively, except where otherwise indicated.

Neither the title nor any abstract of the present disclosure should be taken as limiting in any way the scope of the claimed invention.

Where the preamble of a claim recites a purpose, benefit or possible use of the claimed invention, it does not necessarily limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean either that two or more elements are in direct physical or causal contact, or that two or more elements are not in direct contact with each other yet still co-operate or interact with each other.

It will be appreciated by persons skilled in the art that numerous variations or modifications may be made to the present technology as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present technology. The disclosed implementations are, therefore, to be considered in all respects as illustrative and not limiting or restrictive.

The features described in relation to one or more aspects of the present technology are to be understood as applicable to other aspects of the present technology. More generally, combinations of the steps in the method(s) of the present technology or the features of the system(s) or device(s) of the present technology described elsewhere in the present disclosure, including in the claims, are to be understood as falling within the scope of the present disclosure.

### INDUSTRIAL APPLICABILITY

It is apparent from the above that the arrangements described are applicable to the health care industries.

### LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| ground | 130 |
| array | 150 |
| biphasic stimulus pulse | 160 |
| ECAPs | 170 |
| target fibres | 180 |
| communications channel | 190 |
| external computing device | 192 |
| CLNS system | 300 |
| clinical settings controller | 302 |
| target ECAP controller | 304 |
| box | 308 |
| box | 309 |
| controller | 310 |
| box | 311 |
| stimulator | 312 |
| stimulator | 312A |
| stimulator | 312B |
| stimulator | 312C |
| stimulator | 312D |
| summing element | 313 |
| measurement circuitry | 318 |
| signal window | 319 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| target ECAP amplitude | 520 |
| ECAP | 600 |
| neural stimulation system | 700 |
| neuromodulation device | 710 |
| remote controller | 720 |
| CST | 730 |
| CI | 740 |
| charger | 750 |
| program | 800 |
| stimulus pulse | 810 |
| inter - stimulus interval | 815 |
| stimulus pulse | 820 |
| stimulus pulse | 830 |
| stimulus pulse | 840 |
| stimulus pulse | 850 |
| ECAP | 860 |
| stimulus period | 890 |
| multi - stimset CLNS system | 900 |
| electrode array | 1000 |
| return electrode | 1010a |
| return electrode | 1010b |
| electrode | 1020 |
| measurement electrodes | 1030 |
| box | 1035 |
| return electrode | 1040a |
| return electrode | 1040b |
| stimulus electrode | 1045 |
| electrode array | 1050 |
| electrode | 1060a |
| return electrode | 1060b |
| stimulus electrode | 1070 |
| measurement electrodes | 1080 |
| box | 1085 |
| return electrode | 1090a |
| return electrode | 1090b |
| stimulus electrode | 1095 |
| multi - stimset program | 1100 |
| pulse | 1110 |
| inter - stimulus interval | 1115 |
| pulse | 1120 |
| evoked neural response | 1130 |
| signal window | 1135 |
| shorting pulse | 1140 |
| fixed interval | 1145 |
| stimulus pulse | 1150 |
| shorting pulse | 1160 |
| multi - stimset program | 1200 |
| pulse | 1210 |
| inter - stimulus interval | 1215 |
| pulse | 1220 |
| evoked neural response | 1230 |
| signal window | 1235 |
| shorting pulse | 1240 |
| predetermined interval | 1245 |
| multi - stimset program | 1300 |
| stimulus pulse | 1310 |
| stimulus pulse | 1320 |
| stimulus pulse | 1330 |
| stimulus pulse | 1340 |
| ECAP | 1360 |
| stimulus pulse | 1400 |
| duration | 1410 |
| duration | 1420 |
| duration | 1430 |
| graph | 1500 |
| trace | 1510 |
| trace | 1520 |
| trace | 1530 |
| trace | 1540 |
| trace | 1550 |
| multi - stimset program | 1600 |
| pulse | 1610 |
| inter - stimulus interval | 1615 |
| pulse | 1620 |
| neural response | 1630 |
| signal window | 1635 |
| shorting pulse | 1640 |
| fixed interval | 1645 |
| stimulus pulse | 1650 |
| shorting pulse | 1660 |
| program | 1700 |
| stimulus pulse | 1710 |
| charge balance phase | 1715 |
| stimulus phase | 1720 |
| neural response | 1730 |
| signal window | 1735 |
| shorting pulse | 1740 |
| shorting pulse | 1745 |
| stimulus pulse | 1750 |
| program | 1760 |
| stimulus pulse | 1770 |
| shorting pulse | 1780 |
| stimulus pulse | 1790 |

1. An implantable device for controllably delivering neural stimuli, the device comprising:
a pulse generator configured to deliver neural stimuli, according to a stimulation set, to a neural pathway of a patient, the neural stimuli being configured to evoke neural responses from the neural pathway, wherein the stimulation set defines one or more implanted stimulation electrodes and one or more stimulus parameters;
measurement circuitry configured to capture a signal sensed on the neural pathway, during a signal window, by one or more implanted measurement electrodes; and
a controller configured to:
electrically connect together the stimulation electrodes defined by a first stimulation set and the stimulation electrodes defined by a second stimulation set for a shorting interval of time;
control the pulse generator to deliver a first neural stimulus according to the first stimulation set;
control the pulse generator to deliver a second neural stimulus according to the second stimulation set;
control the measurement circuitry to capture a signal sensed on the neural pathway during a signal window subsequent to delivery of the second neural stimulus; and
measure, from the signal, a characteristic of a neural response evoked by the second neural stimulus.

2. The device of claim 1, wherein the controller is further configured to:
adjust, using a feedback controller, a stimulus parameter of the one or more stimulus parameters defined by the second stimulation set based on the measured characteristic.

3. The device of claim 2, wherein the controller is configured to repeatedly carry out the connecting, controlling, measuring, and adjusting so as to maintain the measured characteristic at or near a target value.

4. The device of claim 2, wherein the controller is further configured to adjust a stimulus parameter of the one or more stimulus parameters defined by the first stimulation set based on the adjustment to the stimulus parameter of the one or more stimulus parameters defined by the second stimulation set.

5. The device of claim 1, wherein the first neural stimulus is an asymmetric triphasic stimulus pulse.

6. The device of claim 1, wherein the electrically connecting together comprises electrically connecting together all the implanted stimulation electrodes.

7. A method of controllably delivering neural stimuli to a neural pathway of a patient via a plurality of implanted stimulation electrodes, the method comprising:
electrically connecting together the stimulation electrodes defined by a first stimulation set and the stimulation electrodes defined by a second stimulation set for shorting interval of time, wherein each stimulation set defines one or more the implanted stimulation electrodes and one or more stimulus parameters;
delivering a first neural stimulus to the neural pathway of the patient, the neural stimulus being delivered according to the first stimulation set;
delivering a second neural stimulus to the neural pathway of the patient, the neural stimulus being delivered according to the second stimulation set;
capturing a signal sensed on the neural pathway during a signal window subsequent to delivering the second neural stimulus; and
measuring, from the signal a characteristic of a neural response evoked by the second neural stimulus.

8. The method of claim 7, further comprising adjusting a stimulus parameter of the one or more stimulus parameters defined by the second stimulation set based on the measured characteristic.

9. The method of claim 8, comprising repeatedly carrying out the connecting, delivering, capturing, measuring, and adjusting so as to maintain the measured characteristic at or near a target value.

10. The method of claim 8, further comprising adjusting a stimulus parameter of the one or more stimulus parameters defined by the first stimulation set based on the adjustment to the stimulus parameter of the one or more stimulus parameters defined by the second stimulation set.

11. The method of claim 7, wherein the first neural stimulus is an asymmetric triphasic stimulus pulse.

12. The method of claim 7, wherein the connecting together comprises connecting together all the implanted electrodes.
